SEQUENCE LISTING (10 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, synthetic construct
  REGION 1..595: note = AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, synthetic construct
  REGION 1..595: note = AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, synthetic construct
  REGION 1..595: note = AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, synthetic construct
  REGION 1..595: note = AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, synthetic construct
  REGION 1..595: note = AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

(19) United States
(12) Patent Application Publication
(10) Pub. No.: US 2026/0053150 A1
(43) Pub. Date: Feb. 26, 2026

(54) Baking with Thermostable AMG Glucosidase Variants (EC 3.2.1.3) and Low Added Sugar

(71) Applicant: Novozymes A/S, Bagsvaerd (DK)

(72) Inventors: Henrik LUNDKVIST, Bagsvaerd (DK); Camilla VARMING, Bagsvaerd (DK)

(73) Assignee: Novozymes A/S, Bagsvaerd (DK)

(21) Appl. No.: 19/105,772

(22) PCT Filed: Nov. 30, 2022
(86) PCT No.: PCT/EP2022/083876

§ 371 Date: Feb. 21, 2025

(30) Foreign Application Priority Data
Sep. 1, 2022 (EP) 22193510.9

(51) Int. Cl. A21D 8/04; C12N 9/34

(52) U.S. Cl. CPC A21D 8/042; C12N 9/2428; C12Y 302/01003

(57) ABSTRACT

The invention relates to methods of producing a baked product, said method comprising adding a mature thermostable variant of a parent glucoamylase at least 70% identical to SEQ ID NO:1, SEQ ID NO:6, SEQ ID NO:7, SEQ ID NO:8 or SEQ ID NO:10 to a dough, adding less than 150% (baker's %) of sugar to the dough, and baking the dough as well of uses of a mature 5 thermostable variant of a parent glucoamylase at least 70% identical to SEQ ID NO:1, SEQ ID NO:6, SEQ ID NO:7, SEQ ID NO:8 or SEQ ID NO:10 for producing a baked product, wherein the variant is added to a dough, less than 150% (baker's %) of sugar or sucrose is added to the dough, and the dough is baked.

### REFERENCE TO SEQUENCE LISTING

This application contains a Sequence Listing in computer readable form, which is incorporated herein by reference.

### FIELD OF THE INVENTION

The invention relates to methods of producing a baked product with a reduced amount of added sugar compared with a standard recipe, said method comprising adding a mature thermostable variant of a parent glucoamylase at least 70% identical to SEQ ID NO:1, SEQ ID NO:6, SEQ ID NO:7, SEQ ID NO:8 or SEQ ID NO:10 to a dough, adding a reduced amount of sugar compared with the standard recipe, and baking the dough.

### BACKGROUND OF THE INVENTION

The recipes of different baked goods can vary to a great extent. One ingredient is sugar, sugar impacts the baking process as well as the final baked goods.
The dough properties: making the dough softer with increasing sugar levels
Provides food for the yeast. However, also inhibiting the yeast activity giving less gassing power
Improving the crust coloration giving the final bread a more golden crust color.
Impacting the crumb of the baked goods and keeps it soft and moist.
Gives the baked product a sweet flavor.

There is a trend in the market to reduce the amount of sugar in food. Taking out the sugar will impact the baking process and the final baked product. Making the dough more firm, generating less crust coloration during baking, generating a less soft and moist product and making the baked product less sweet.

Enzymes can improve some of these properties. The crust coloration can be improved with a glucoamylase like Goldcrust®. The softness of the crumb can be improved by maltogenic amylases like Novamyl® G, Novamyl 3D® G, Novamyl® Pro.

Recipe changes can improve other parameters that is impacted by taking out sugar. Increasing the water content can make the dough softer. The increased gassing power of the yeast when taking out sugar can be adjusted by reducing the yeast level.

### SUMMARY OF THE INVENTION

The inventors found that mature thermostable glucoamylase variants showed increased enzyme activity or performance in baking recipes having a lower starting concentration of added sugar. An increased enzyme activity may translate into a lower enzyme dosage compared with recipes having a higher starting concentration of added sugar. The improved activity of the thermostable variants resulted in increased sweetness or sweet taste of the product, because glucoamylase produce glucose. This finding enables a reduction in the amount of added sugar in traditional baking recipes without any perceived loss in sweetness in the final baked product, which is very much of commercial interest. At the same time the recipe can be adjusted by adding more water and reducing the yeast level generating more cost savings.

Accordingly in a first aspect, the invention relates to methods of producing a baked product, said method comprising adding a mature thermostable variant of a parent glucoamylase at least 70% identical to SEQ ID NO:1, SEQ ID NO:6, SEQ ID NO:7, SEQ ID NO:8 or SEQ ID NO:10 to a dough, adding less than 150% (baker's %) of sugar to the dough, and baking the dough.

A second aspect of the invention relates to uses of a mature thermostable variant of a parent glucoamylase at least 70% identical to SEQ ID NO:1, SEQ ID NO:6, SEQ ID NO:7, SEQ ID NO:8 or SEQ ID NO:10 for producing a baked product, wherein the variant is added to a dough, less than 150% (baker's %) of sugar or sucrose is added to the dough, and the dough is baked.

Preferably, the method of the first aspect or the use of the second aspect comprises adding a mature thermostable variant of a parent glucoamylase at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO:1, SEQ ID NO:6, SEQ ID NO:7, SEQ ID NO:8 or SEQ ID NO:10.

A preferred embodiment of the invention relates to adding less than 120% (baker's %) of sugar to the dough, preferably less than 110% (baker's %), preferably less than 100% (baker's %), preferably less than 90% (baker's %), preferably less than 80% (baker's %), preferably less than 70% (baker's %), preferably less than 60% (baker's %), preferably less than 50% (baker's %), preferably less than 40% (baker's %), preferably less than 30% (baker's %), preferably less than 20% (baker's %), preferably less than 10% (baker's %), preferably less than 8% (baker's %), preferably less than 6% (baker's %), even more preferably less than 4% (baker's %) and most preferably less than 2% (baker's %) of sugar to the dough.

### FIGURES

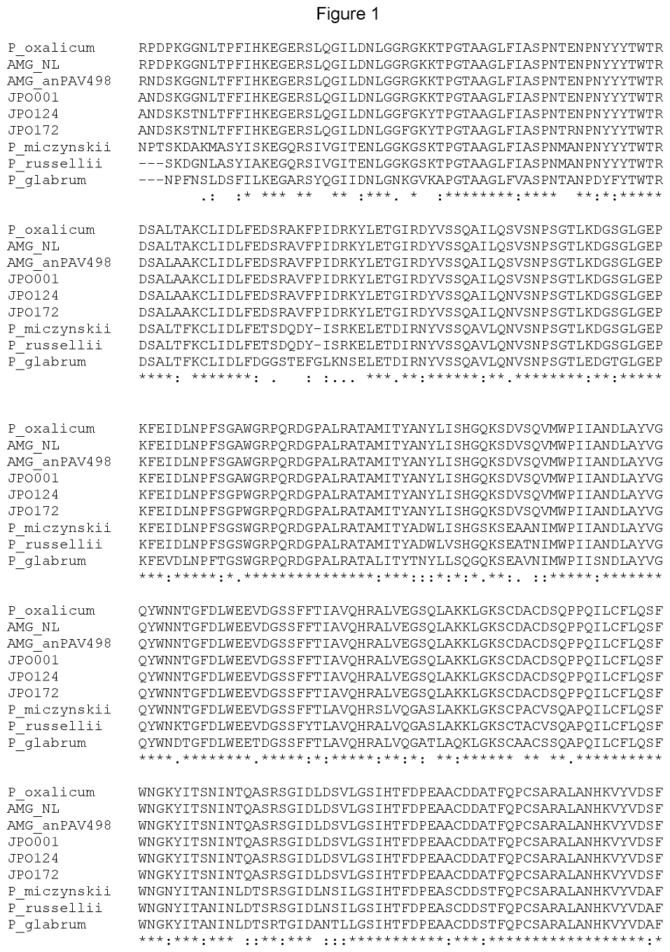

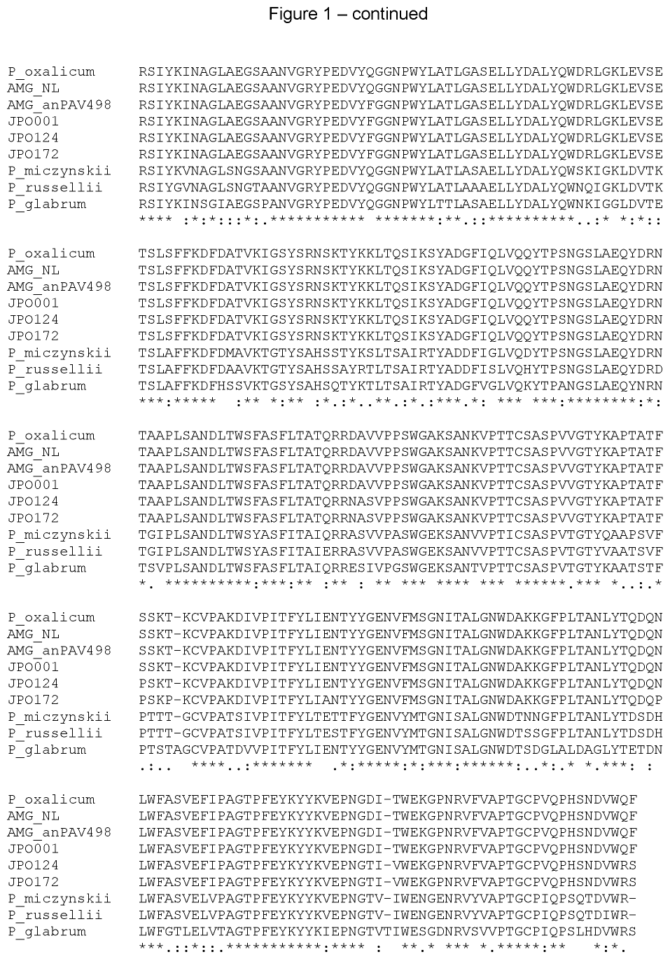

FIG. 1 shows a multiple alignment of the amino acid sequences of the mature proteins of:
Wildtype AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO-172’ of SEQ ID NO:6
Wildtype AMG from Penicillium miczynskii (PoAMG) of SEQ ID NO:7
Wildtype AMG from Penicillium russellii (PoAMG) of SEQ ID NO:8
Wildtype AMG from Penicillium glabrum (PoAMG) of SEQ ID NO:9

### DETAILED DESCRIPTION OF THE INVENTION

### Definitions

Baked product: As used herein, “baked product” means any kind of baked product including bread types such as pan bread, toast bread, open bread, pan bread with and without lid, buns, hamburger buns, rolls, baguettes, brown bread, whole meal bread, rich bread, bran bread, flat bread, tortilla, pita, Arabic bread, Indian flat bread, cookies, biscuits, cakes, brioche and any variety thereof.

Dough: As used herein “dough” means any dough used to prepare a bread. The dough used to prepare a baked product may be made from any suitable dough ingredients, including flour sourced from grains, such as, wheat flour, corn flour, rye flour, barley flour, oat flour, rice flour, or sorghum flour, potato flour, soy flour, and combinations thereof (e.g., wheat flour combined with one of the other flour sources; rice flour combined with one of the other flour sources). The dough of the invention is normally a leavened dough or a dough to be subjected to leavening. The dough may be leavened in various ways, such as by adding dough ingredients such as chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae. The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulphate, calcium sulphate, diluents such silica dioxide, starch of different origins. Still other convention ingredients include hydrocolloids such as CMC, guar gum, xanthan gum, locust bean gum, etc. Modified starches may be also used. The dough ingredients may comprise fat (triglyceride) such as granulated fat or shortening, but the invention is particularly applicable to a dough where less than 1% by weight of fat or shortening is added, and particularly to a dough which is made without addition of fat or shortening. In a preferred embodiment, the dough ingredients comprise wheat flour; preferably 10% (w/w) or more of the total flour content is wheat flour, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or preferably at least 95% (w/w) of the flour is wheat flour. The dough may be prepared applying any conventional mixing process, such as the continuous mix process, straight-dough process, or the sponge and dough method.

Baker's %: Baker's percent is a mathematical method widely used in baking to calculate the amounts of macro, minor and micro ingredients. It's based on the total weight of flour a formula contains. Instead of dividing each ingredient's weight by the total formula weight, bakers divide each ingredient by the weight of flour.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the -no brief option) is used as the percent identity and is calculated as follows:

$$( Identical Residues \times 100 ) / ( Length of Alignment - Total Number of Gaps in Alignment )$$

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Thermostability improvement: The thermostability improvement (Td) in ° C. is a measure of how much the variants have improved in thermostability over their parent glucoamylase under the same conditions, determined as exemplified herein.

As used herein the term “grist” is understood as the starch or sugar containing material that is the basis for beer production, e.g. the barley malt and the adjunct. Generally, the grist does not contain any added water.

The term “starch gelatinization” is understood as the irreversible order-disorder transition that starch undergoes when heated in the presence of water. Differential Scanning calorimetry (DSC) in one technique that can be employed to study the gradual process of starch gelatinization describing the onset and peak temperature (Tₒ & Tₚ) of starch gelatinization. The term “onset gelatinization temperature (Tₒ)” is understood as the temperature at which the gelatinization begins. The term “peak gelatinization temperature (Tₚ)” is understood as the temperature at endotherm peak. The term “conclusion gelatinization temperature (T_{c})” is understood as the temperature at which the gelatinization has terminated.

### Glucoamylases

Glucoamylases are also called amyloglucosidases, and Glucan 1,4-alpha-glucosidase (EC 3.2.1.3), more commonly they are referred to as AMGs.

According to the present invention, different types of amyloglucosidases may be used as parent for the generation of a thermostable amyloglucosidase variant, e.g, the amyloglucosidase may be a polypeptide that is encoded by a DNA sequence that is found in a fungal strain of Aspergillus, Rhizopusor, Talaromyces (Rasamsonia) or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Preferably, the parent glucoamylase is from a species of Penicillium, preferably from Penicillium oxicalum, Penicillium miczynskii, Penicillium russellii or Penicillium glabrum.

Examples of other suitable fungi include Aspergillus niger, Aspergillus awamori, Aspergillus oryzae, Rhizopus delemar, Rhizopus niveus, Rhizopus oryzae and Talaromyces emersonii (Rasamsonia emersonii).

Below is shown the %-identity between the AMG amino acid sequences aligned in FIG. 1, and also provided in the sequence list:

| | | | | | | | | | |
|---|---|---|---|---|---|---|---|---|---|
| P_oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO-172 | 95.97 | 96.13 | 96.97 | 97.14 | 99.33 | 100.00 | 76.73 | 76.78 | 73.99 |
| P_miczynskii | 77.07 | 77.07 | 76.73 | 76.73 | 77.07 | 76.73 | 100.00 | 94.75 | 80.51 |
| P_russellii | 77.12 | 77.12 | 76.95 | 76.95 | 77.12 | 76.78 | 94.75 | 100.00 | 79.66 |
| P_glabrum | 74.32 | 74.32 | 73.82 | 73.82 | 74.32 | 73.99 | 80.51 | 79.66 | 100.00 |

Thermostable variants of the PoAMG have been generated (see table 2 below). In a preferred embodiment, the mature thermostable glucoamylase variant of the invention comprises one or more or all of the combinations of amino acid substitutions listed in table 2 below.

In a preferred embodiment, the mature variant of the invention comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO:1; preferably the at least one amino acid modification comprises a substitution in one or more or all of the positions corresponding to positions 1, 2, 4, 11, 65, 79 and 327 in SEQ ID NO:1, preferably the at least one amino acid modification comprises a substitution in one or more or all of the positions corresponding to R1A, P2N, P4S, P11F, T65A, K79V and Q327F in SEQ ID NO:1; or preferably the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

The thermostability improvements (Td) of the variants in table 2 are listed in Table 3, where the Td of the PoAMG variant denoted “anPAV498” (the parent) was set to zero. In a preferred embodiment, the mature thermostable variant of the invention has a thermostability improvement (Td) over its parent of at least 5° C., preferably at least 6° C., 7° C. or 8° C., preferably determined as exemplified herein.

In another preferred embodiment, the mature thermostable variant of the invention has a relative activity at 91° C. of at least 150, preferably at least 200, more preferably at least 250, most preferably at least 300 compared to its parent.

### Additional Enzymes

In a preferred embodiment of the first aspect, one or more additional enzyme is added to the dough, said additional enzyme may be selected from the group consisting of a alpha amylase, maltogenic amylase, raw-starch degrading alpha amylase, beta amylase, aminopeptidase, carboxypeptidase, catalase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, raw starch degrading alpha amylase, ribonuclease, transglutaminase, and xylanase.

### Lipases

In a preferred embodiment of the first aspect, one or more additional enzyme is added, said additional enzyme is preferably a mature lipolytid enzyme, preferably a mature lipolytic enzyme disclosed in WO 2018/150021 (Novozymes A/S), more preferably a mature polypeptide having lipolytic enzyme activity and having at least 65% sequence identity to amino acids 21 to 309 of SEQ ID NO:1 in WO 2018/150021, or a polypeptide encoded by a polynucleotide having at least 65% sequence identity to the mature polypeptide coding sequence of SEQ ID NO 2 in WO 2018/150021.

### Raw Starch Degrading Alpha-Amylase

As used herein, a “raw starch degrading alpha-amylase” refers to an enzyme that can directly degrade raw starch granules below the gelatinization temperature of starch.

Examples of raw starch degrading alpha-amylases include the ones disclosed in WO 2005/003311, U.S. Patent Publication no. 2005/0054071, and U.S. Pat. No. 7,326,548. Examples also include those enzymes disclosed in Table 1 to 5 of the examples in U.S. Pat. No. 7,326,548, in U.S. Patent Publication no. 2005/0054071 (Table 3 on page 15), as well as the enzymes disclosed in WO 2004/020499 and WO 2006/06929 and WO 2006/066579.

In one embodiment, the raw starch degrading alpha-amylase is a GH13_1 amylase.

In one embodiment, the raw starch degrading alpha-amylase enzyme has at least 70%, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identity to the raw starch degrading alpha-amylase shown in EP U.S. Pat. No. 2,981,170 (Novozymes A/S) or in SEQ ID NO:11 herein.

### Amylases

Alpha-Amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

A number of alpha-amylases are referred to as Termamyl™, Termamyl® SC and “Termamyl™-like alpha-amylases” and are known from, e.g., WO 90/11352, WO 95/10603, WO 95/26397, WO 96/23873 and WO 96/23874.

Another group of alpha-amylases are referred to as Fungamyl™ and “Fungamyl™-like alpha-amylases”, which are alpha-amylases related to the alpha-amylase derived from Aspergillus oryzae disclosed in WO 01/34784.

### Proteases

Suitable proteases include microbial proteases, such as fungal and bacterial proteases. Preferred proteases are acidic proteases, i.e., proteases characterized by the ability to hydrolyze proteins under acidic conditions below pH 7. The proteases are responsible for reducing the overall length of high-molecular-weight proteins to low-molecular-weight proteins in the mash. The low-molecular-weight proteins are a necessity for yeast nutrition and the high-molecular-weight-proteins ensure foam stability. Thus it is well-known to the skilled person that protease should be added in a balanced amount which at the same time allows ample free amino acids for the yeast and leaves enough high-molecular-weight-proteins to stabilize the foam. In one aspect, the protease activity is provided by a proteolytic enzymes system having a suitable FAN generation activity including endo-proteases, exopeptidases or any combination hereof, preferably a metallo-protease. Preferably, the protease has at least 50%, more preferably at least 60%, more preferably at least 70%, more preferably at least 80%, more preferably at least 85%, more preferably at least 90%, more preferably at least 91%, more preferably at least 92%, more preferably at least 93%, more preferably at least 94%, more preferably at least 95% more preferably at least 96%, more preferably at least 97% more preferably at least 98%, and most preferably at least 99% or even 100% identity to the amino acid sequence shown in SEQ ID NO:6 described in WO9967370. In another aspect, the protease is Neutrase® available from Novozymes A/S. Proteases may be added in the amounts of, 0.0001-1000 AU/kg DS, preferably 1-100 AU/kg DS and most preferably 5-25 AU/kg dry weight cereal(s). The proteolytic activity may be determined by using denatured hemoglobin as substrate. In the Anson-Hemoglobin method for the determination of proteolytic activity, denatured hemoglobin is digested, and the undigested hemoglobin is precipitated with trichloroacetic acid (TCA). The amount of the TCA soluble product is determined by using phenol reagent, which gives a blue color with tyrosine and tryptophan. One Anson Unit (AU) is defined as the amount of enzyme which under standard conditions (i.e. 25° C., pH 7.5 and 10 min. reaction time) digests hemoglobin at an initial rate such that there is liberated an amount of TCA soluble product per minute which gives the same colour with phenol reagent as one milliequivalent of tyrosine.

### Enzyme Compositions

The mature thermostable variant glucoamylase of the invention as well as any additional enzyme(s) may be added in any suitable form, such as, e.g., in the form of a liquid, in particular a stabilized liquid, or it may be added as a substantially dry powder or granulate.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added in any suitable manner, such as individual components (separate or sequential addition of the enzymes) or addition of the enzymes together in one step or one composition.

Granulates and agglomerated powders may be prepared by conventional methods, e.g., by spraying the enzymes onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

### EXAMPLES

### Example 1: Construction of PoAMG Libraries

PoAMG Libraries were Constructed as Follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing JPO-0001 gene) by the following conditions. The resultant PCR fragments were purified by QIAquick Gel extraction kit [QIAGEN], and then introduced into Escherichia coli ECOS Competent E. coli DH5a [NIPPON GENE CO., LTD.]. The plasmid DNAs were extracted from E. coli transformants by MagExtractor plasmid extraction kit [TOYOBO], and then introduced into A. niger competent cells.

### PCR Reaction Mix:

PrimeSTAR Max DNA polymerase [TaKaRa]
Total 25 μl

| | |
|---|---|
| 1.0 μl | Template DNA (1 ng/μl) |
| 9.5 μl | H₂O |
| 12.5 μl | 2x PrimeSTAR Max pre-mix |
| 1.0 μl | Forward primer (5 μM) |
| 1.0 μl | Reverse primer (5 μM) |

### PCR Program:

98° C./2 min
25× (98° C./10 sec, 60° C./15 sec, 72° C./2 min)
10° C./hold

### Example 2: Screening for Better Thermostability

B. subtilis libraries constructed as in EXAMPLE 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5N NaOH, 10 ml/L COVE salt, 10 ml/L 1M acetamide), 32° C. for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70° C. for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| Name | Relative activity of 80° C. / 75° C. (%) |
|---|---|
| anPAV498 | 17% |
| JPO-004 | 32% |
| JPO-005 | 15% |
| JPO-006 | 16% |
| JPO-007 | 3% |
| AnPav498 | 13% |
| JPO-009 | 16% |
| JPO-011 | 15% |
| JPO-012 | 15% |
| JPO-013 | 17% |
| JPO-020 | 20% |

| Name | Relative activity of 80° C. / 70° C. (%) |
|---|---|
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |
| Name | Relative activity of 79° C. / 70° C. (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |
| Name | Relative activity of 77° C. / 70° C. (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |
| name | Relative activity of 79° C. / 77° C. (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| Name | Relative activity of 77° C. / 70° C. (%) |
|---|---|
| JPO-022 | 60% |
| JPO-027 | 67% |
| JPO-042 | 8% |
| JPO-044 | 86% |
| JPO-045 | 67% |
| JPO-046 | 48% |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |
| Name | Relative activity of 79° C. / 77° C. (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
Lists of the relative activity of PoAMG variants when compared
with their parent JPO-063 at different temperatures

| Name | Relative activity of 79° C. / 77° C. (%) |
|---|---|
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |
| Name | Relative activity of 84° C. / 80° C. (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |
| Name | Relative activity of 82° C. / 70° C. (%) |
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |
| Name | Relative activity of 83° C. / 80° C. (%) |
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| Name | Relative activity of 83° C. / 70° C. (%) |
|---|---|
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |
| Name | Relative activity of 83° C. / 80° C. (%) |
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| Name | Relative activity of 84° C. / 80° C. (%) |
|---|---|
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| Name | Relative activity of 84° C. / 75° C. (%) |
|---|---|
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105L A132R D445N V447S Y504T D566T Q594R F595S |
| JPO-064 | R1A K34Y S103N S105L D445N V447S D566T Q594R F595S |
| JPO-065 | R1A K34Y S103N S105L A132R D445N V447S E501V Y504T D566T Q594R R1A |
| | F595S |
| JPO-066 | R1A K34Y S103N A132R D445N V447S Y504T D566T Q594R F595S |
| JPO-069 | R1A K34Y S103N S105L A132R D445N V447S Y504T D566T V592T |
| JPO-071 | R1A G6S G7T K34Y S103N S105L A132R D445N V447S Y504T D566T Q594R |
| | R1A F595S |
| JPO-074 | R1A K34Y S103N P107L A132R D445N V447S Y504T D566T Q594R F595S |
| JPO-083 | R1A G6S G7T K34Y S103N P107L A132R D445N V447S Y504T D566T Q594R |
| | R1A F595S |
| JPO-084 | R1A G6S G7T K34Y S103N P107L A132R D445N V447S Y504T D566T V592T |
| | R1A Q594R F595S |
| JPO-091 | R1A G6S R7T K34Y S103N P107L A132P D445N V447S Y504T D566T Q594R |
| | F595S |
| JPO-092 | R1A G6S G7T K34Y S103N P107L A132R D445N V447S Y504T D566T T568V |
| | Q594R F595S |
| JPO-093 | R1A G6S G7T K34Y S103N P107L A132P D445N V447S Y504T D566T T568V |
| | Q594R F595S |
| JPO-094 | R1A G6S G7T K34Y S103N P107L A132R D445N V447S S481P Y504T D566T |
| | Q594R F595S |
| JPO-095 | R1A G6S G7T K34Y S103N P107L A132R D445N V447S S481P Y504T D566T |
| | T568V Q594R F595S |
| JPO-096 | R1A G6S G7T K34Y S103N P107L A132P D445N V447S D566T T568V Q594R |
| | F595S |
| JPO-097 | R1A G6S G7T K34Y S103N P107L T110W A132P D445N V447S Y504T D566T |
| | T568V Q594R F595S |
| JPO-098 | R1A G6S G7T K34Y E50R S103N P107L A132P D445N V447S Y504T D566T |
| | T568V Q594R F595S |
| JPO-105 | R1A G6S G7T K34Y S103N P107L A132P D445N V447S E501V Y504T |
| JPO-106 | R1A G6S G7T R31F K34Y S103N P107L A132P D445N V447S Y504T D566T |
| | T568V Q594R F595S |
| JPO-108 | R1A G6S G7T R31F K34Y S103N P107L A132P D445N V447S S481P Y504T |
| | D566T T568V Q594R F595S |
| JPO-109 | R1A G6S G7T K34Y E50R S103N P107L A132P D445N V447S S481P Y504T |
| | D566T T568V Q594R F595S |
| JPO-111 | R1A G6S G7T R31F K34Y S103N P107L A132P D445N V447S S481P E501V |
| | Y504T D566T T568V Q594R F595S |
| JPO-112 | R1A G6S G7T R31F K34Y S103N P107L A132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107L A132P D445N |
| | V447S S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### Example 3: Fermentation of the Aspergillus niger

Aspergillus niger strains were fermented on a rotary shaking table in 500 ml baffled flasks containing 100 ml MU1 with 4 ml 50% urea at 220 rpm, 30° C. The culture broth was centrifuged (10,000×g, 20 min) and the supernatant was carefully decanted from the precipitates.

### Example 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### Example 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02° C./sec
Scan range: 37-96° C.
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### Example 6: PoAMG Activity Assay

Maltodextrin (DE11) assay by GOD-POD method

### Substrate Solution

30 g maltodextrin (pindex #2 from MATSUTANI chemical industry Co., Ltd.)
100 ml 120 mM sodium acetate buffer, pH 5.0

### Glucose CII Test Kit (Wako Pure Chemical Industries, Ltd.)

Twenty μl of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl PH 8.0 to stop reaction. Ten ul of the solution was mixed with 200 μl of the working solution of the test kit then stand at room temperature for 15 min. The absorbance at A505 was read. The activities are listed in Table 3 as relative activity of the PoAMG variant denoted anPAV498.

TABLE 3

| | Td improvement [° C.] | Activity at 91° C. |
|---|---|---|
| Variant | (pH 5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: Effect of Reducing Sugar (Sucrose) Levels on the Efficiency of JPO-172 in Straight Dough Bread

Breads were baked in a straight dough baking process with a recipe according to Table 4. Different treatments were made according to Table 5. Breads with lower levels of sucrose had 2.5% additional water in order to have the same dough properties of all doughs. The bread was baked in lidded tins in order to have the same volume of all bread. The ingredients were mixed in a spiral mixer into a dough for 3 min at 17 rpm and 7 min at 35 rpm. The doughs were allowed to rest for 5 minutes and divided into 450 g dough pieces and rounded. The dough pieces were sheeted and placed in baking tins. The tins with the doughs were proofed for 55 min at 32° C. and 86% relative humidity. The proofed doughs were baked in a deck oven for 28 min at 230° C.

TABLE 4
Recipe

| | Bakers % |
|---|---|
| Flour (Kolibri, Meneba, NL) | 100 |
| Water | Variable |
| Yeast | 5 |
| Sucrose | Variable |
| Salt | 1.8 |
| Ascorbic acid | 0.04 |
| Calcium Propionate | 0.425 |
| Fungamyl ® G | 10 ppm |
| Panzea ® G | 25 ppm |

TABLE 5
Treatments

| | 1 | 2 | 3 | 4 | 5 | 6 |
|---|---|---|---|---|---|---|
| Sucrose, % | 8 | 8 | 2 | 2 | 2 | 2 |
| Water, % | 54.5 | 54.5 | 57 | 57 | 57 | 57 |
| JPO-172, | | 46 | 38 | 35 | 31 | 27 |
| mgEP/kg flour | | | | | | |

The bread was packed 2 hours after baking in sealed plastic bags and stored at room temperature until analysis.

The texture of the bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 6 and Table 7. Bread without JPO-172 enzyme (Control) is soft and elastic on day 1 and when the bread is stored for 14 days it becomes more firm and less elastic with time. When 46 mgEP JPO-172 enzyme per kg flour is added, the firmness on day 1 is reduced from 496 g to 285 g and the elasticity increases from 57.4% to 66.5%. As the bread with JPO-172 is stored for 14 days it becomes more firm and less elastic. However, not nearly to the same extent as bread without JPO-172.

The JPO-172 enzyme becomes even more efficient if the sucrose level of the bread is decreased from 8% to 2%. A dosage of 31 mgEP/kg flour when there is 2% sucrose in the recipe gives a firmness and elasticity that are not significantly different compared to 46 mgEP/kg flour when there is 8% sucrose in the recipe on day 1 to day 14. A higher dosage of JPO-172 (38 mgEP/kg flour) when there is 2% sucrose in the recipe will be less firm on day 7 and 14 compared to a 46 mgEP/kg flour when there is 8% sucrose in the recipe.

TABLE 6
Firmness (g) of bread crumb at different timepoints. The
letter behind the number represents significance levels
using a student t-test with a significance level of 0.05.

| Treatment | Day 1 | Day 7 | Day 14 |
|---|---|---|---|
| Control (8% sucrose) | 496 A | 1094 A | 1579 A |
| 46 mgEP/kg flour JPO-172 (8% sucrose) | 285 B | 614 BC | 913 C |
| 27 mgEP/kg flour JPO-172 (2% sucrose) | 284 B | 640 B | 1040 B |
| 31 mgEP/kg flour JPO-172 (2% sucrose) | 269 B | 654 B | 885 C |
| 35 mgEP/kg flour JPO-172 (2% sucrose) | 267 B | 565 C | 894 C |
| 38 mgEP/kg flour JPO-172 (2% sucrose) | 230 B | 481 D | 779 D |

TABLE 7
Elasticity (%) of bread crumb at different timepoints. The
letter behind the number represents significance levels
using a student t-test with a significance level of 0.05.

| Treatment | Day 1 | Day 7 | Day 14 |
|---|---|---|---|
| Control (8% sucrose) | 57.4 B | 45.8 E | 43.7 D |
| 46 mgEP/kg flour JPO-172 (8% sucrose) | 66.5 A | 61.0 C | 56.8 B |
| 27 mgEP/kg flour JPO-172 (2% sucrose) | 67.6 A | 58.6 D | 53.0 C |
| 31 mgEP/kg flour JPO-172 (2% sucrose) | 68.1 A | 61.2 C | 56.7 B |
| 35 mgEP/kg flour JPO-172 (2% sucrose) | 67.9 A | 62.6 B | 58.8 A |
| 38 mgEP/kg flour JPO-172 (2% sucrose) | 68.2 A | 64.3 A | 59.6 A |

### Example 8. Effect of Reducing Sugar (Sucrose) Levels on the Efficiency of JPO-172 in Sponge and Dough Bread

Breads were baked in a sponge & dough baking process with a recipe according to Table 8. Different treatments were made according to Table 9. Breads with lower levels of sucrose had additional water in order to have the same dough properties of all doughs. The bread was baked in lidded tins in order to have the same volume of all bread. The ingredients of the sponge were mixed in a Pin mixer (Bjørn mixer) into a sponge for 2 min at 50 rpm and 2 min at 150 rpm. The sponge was fermented for 3 hours at 27° C. and 75 rH %. The sponge and the ingredients of the dough were placed in the bowl of the pin mixer and mixed for 1 min at 50 rpm and 3 min at 150 rpm. The doughs were divided into 400 g dough pieces rounded and allowed to rest for 5 minutes. The dough pieces were sheeted and placed in baking tins. The tins with the doughs were proofed for 60 min at 43° C. and 80% relative humidity. The proofed doughs were baked in a revolving oven for 20 min at 215° C.

TABLE 8
Recipe Sponge and Dough

| | Bakers % |
|---|---|
| Sponge | |
| Flour (Pelikaan, Meneba, NL) | 70 |
| Water | 40.6 |
| Dry Yeast | 1.5 |
| SSL | 0.5 |
| Oil (Soybean) | 3 |
| Dimodan HP75 | 02 |
| Calcium Propionate | 0.1 |
| Dough | |
| Flour (Pelikaan, Meneba, NL) | 30 |
| Water | Variable |
| Dry Yeast | 0.5 |
| Sucrose | Variable |
| Salt | 2 |
| Ascorbic acid | 0.06 |
| Calcium Propionate | 0.3 |
| Fungamyl ® G | 10 ppm |
| Panzea ® G | 25 ppm |

TABLE 9
Treatments

| | 1 | 2 | 3 | 4 | 5 |
|---|---|---|---|---|---|
| Water, % | 56.5 | 57.6 | 58.1 | 58.5 | 59 |
| Sucrose, % | 8 | 5 | 4 | 3 | 2 |
| JPO-172, | 0 | 100 | 100 | 100 | 100 |
| mgEP/kg flour | | | | | |

The bread was packed 2 hours after baking in sealed plastic bags and stored at room temperature until analysis.

The texture of the bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 10 and Table 11. A bread without freshness enzyme and 8% sucrose (Treatment 1 Control) is soft and elastic on day 1 and when the bread is stored for 14 days it becomes more firm and less elastic with time.

The addition of the freshness enzyme JPO-172 at a dosage of 100 mgEP/kg flour (Dough 2-5) makes the bread less firm and more elastic in the whole time interval studied (Day 1-14). By reducing the level of sucrose, the freshness enzyme JPO-172 becomes more efficient and can improve the freshness effect even further, thereby making the bread crumb on day 7 and 14 less firm. The improved efficiency of the freshness enzyme by reducing the sucrose level in the recipe can also be seen as increase elasticity in the whole time range studied.

TABLE 10
Firmness (g) of bread crumb at different timepoints. The
letter behind the number represents significance levels
using a student t-test with a significance level of 0.05.

| Treatment | Day 1 | Day 7 | Day 14 |
|---|---|---|---|
| 2% sucrose 100 mgEP/kg JPO-172 | 199 B | 260 C | 323 D |
| 3% sucrose 100 mgEP/kg JPO 172 | 197 B | 304 B | 378 C |
| 4% sucrose 100 mgEP/kg JPO 172 | 201 B | 293 B | 411 BC |
| 5% sucrose 100 mgEP/kg JPO 172 | 191 B | 313 B | 428 B |
| 8% sucrose (Control) | 231 A | 600 A | 875 A |

TABLE 11
Elasticity (%) of bread crumb at different timepoints. The
letter behind the number represents significance levels
using a student t-test with a significance level of 0.05.

| Treatment | Day 1 | Day 7 | Day 14 |
|---|---|---|---|
| 2% sucrose 100 mgEP/kg JPO-172 | 62.5 A | 59.8 A | 57.8 A |
| 3% sucrose 100 mgEP/kg JPO 172 | 61.9 B | 58.8 B | 57.0 B |
| 4% sucrose 100 mgEP/kg JPO 172 | 61.5 C | 58.1 C | 55.7 C |
| 5% sucrose 100 mgEP/kg JPO 172 | 61.2 C | 57.1 D | 54.6 D |
| 8% sucrose (Control) | 52.5 D | 48.0 E | 44.4 E |

### Example 9 Sweetness of Bread with Reduced Levels of Sugar in the Recipe and JPO-172

Breads were baked in a straight dough baking process with a recipe according to Table 12. Different treatments were made according to Table 13. Breads with lower levels of sucrose had 0.3% additional water/% sucrose reduced in order to have the same dough properties of all doughs. Since sugar inhibit the yeast activity the bread with lower level of sugar had 0.25% less yeast/% sugar in order to have the same gassing power of all the doughs. The bread was baked in lidded tins in order to have the same volume of all bread. The ingredients were mixed in a spiral mixer into a dough for 3 min at 17 rpm and 6 min at 35 rpm. The doughs were allowed to rest for 5 minutes and divided into 450 g dough pieces and rounded. The dough pieces were sheeted and placed in baking tins. The tins with the doughs were proofed for 57 min at 32° C. and 86% relative humidity. The proofed doughs were baked in a deck oven for 35 min at 215° C.

TABLE 12
Recipe

| | Bakers % |
|---|---|
| Flour (Kolibri, Meneba, NL) | 100 |
| Water | Variable |
| Yeast | Variable |
| Sucrose | Variable |
| Salt | 1.5 |
| Ascorbic acid | 0.04 |
| Calcium Propionate | 0.25 |
| Fungamyl ® G | 6 ppm |
| Panzea ® G | 25 ppm |

TABLE 13
Treatments

| | 1 | 2 | 3 | 4 | 5 |
|---|---|---|---|---|---|
| Sucrose, % | 8 | 0 | 1 | 2 | 3 |
| Water, % | 54.5 | 56.7 | 56.4 | 56.1 | 55.8 |
| Yeast, % | 5 | 3 | 3.5 | 3.75 | 4 |
| JPO-172 mgEP/kg | | 50 | 50 | 50 | 50 |
| flour | | | | | |

The breads were allowed to cool down at room temperature and packed 2 hours after baking in sealed plastic bags and stored at room temperature until analysis.

### Sensory Evaluation Method:

The sweet taste of the bread was evaluated in a sensory session, day 2 after baking. Each sensory assessor was served 1 slice of each bread type. A training session was held prior to the evaluation defining the sweet taste attribute and procedure (Table 14) and use of intensity scale. Intensity of the sweet taste was evaluated on a 1-9 point intensity scale ranging from little to very intense. The control sample with 8% sucrose was served as reference, with the score 5.0. The other samples were served blind, 3-digit coded, and in random order. Five trained assessors participated in the evaluation. Two sensory replicates were performed. The significance of the sensory results were estimated using a student's t-test with a significance level of 0.05.

TABLE 14
Sensory attribute

| Attribute | Procedure | Evaluate |
|---|---|---|
| Sweet taste | Take a bite of the bread | How sweet the bread taste |
| | and chew it | |

In Table 15 the scores from the sensory evaluation of sweet taste can be found. Even though the doughs with 0-2% added sucrose have less added sugar in the dough compared to the dough with 8% added sucrose, the final baked bread were perceived as sweet as the bread with 8% sucrose in the dough.

TABLE 15
Mean sensory scores of sweet taste. The letters behind
the sensory score represents the statistical significance
using a student's t-test with a = 0.05. Levels
not connected with the same letter are significantly different.

| Sample | Sweet taste |
|---|---|
| Control with 8% sucrose | 5.0 BC |
| JPO-172 with 0% sucrose | 4.7 C |
| JPO-172 with 1% sucrose | 5.2 ABC |
| JPO-172 with 2% sucrose | 5.8 AB |
| JPO-172 with 3% sucrose | 6.2 A |

1. A method of producing a baked product, said method comprising adding a mature thermostable variant of a parent glucoamylase at least 70% identical to SEQ ID NO:1, SEQ ID NO:6, SEQ ID NO:7, SEQ ID NO:8 or SEQ ID NO:10 to a dough, adding less than 150% (baker's %) of sugar to the dough, and baking the dough.

2. The method according to claim 1, wherein the parent glucoamylase is from a species of Penicillium, preferably from Penicillium oxicalum, Penicillium miczynskii, Penicillium russellii or Penicillium glabrum.

3. The method according to claim 1, wherein the mature variant comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO: 1.

4. The method according to claim 3, wherein the at least one amino acid modification comprises a substitution in one or more or all of the positions corresponding to positions 1, 2, 4, 11, 65, 79 and 327 in SEQ ID NO:1.

5. The method according to claim 3, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO: 1.

6. The method according to claim 3, wherein the at least one amino acid modification comprises a substitution in one or more or all of the positions corresponding to positions 1, 6, 7, 31, 34, 50, 103, 132, 445, 447, 481, 501, 539, 566, 568, 594 and 595 in SEQ ID NO: 1.

7. The method according to claim 3, wherein the at least one amino acid modification comprises a substitution in one or more or all of the positions corresponding to R1A, G6S, G7T, R31F, K34Y, E50R, S103N, A132P, D445N, V447S, S481P, E501A, N539P, D566T, T568V, Q594R and F595S in SEQ ID NO: 1.

8. The method according to claim 3, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO: 1.

9. The method according to claim 1, wherein the mature thermostable variant has a thermostability improvement (Td) over its parent of at least 5° C., at least 6° C., 7° C. or 8° C.

10. The method according to claim 1, wherein the mature thermostable variant has a relative activity at 91° C. of at least 150, at least 200, at least 250, or at least 300 compared to its parent.

11. The method according to claim 1, further comprising adding one or more additional enzyme, said additional enzyme selected from the group consisting of a alpha amylase, maltogenic amylase, raw-starch degrading alpha amylase, beta amylase, aminopeptidase, carboxypeptidase, catalase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

12. (canceled)

13. The method according to claim 3, wherein the at least one amino acid modification comprises a substitution in one or more or all of the positions corresponding to R1A, P2N, P4S, P11F, T65A, K79V and Q327F in SEQ ID NO: 1.

14. The method according to claim 3, wherein the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO: 1.

15. The method according to claim 3, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO: 1.
